(19) United States
(12) Patent Application Publication
(10) Pub. No.: US 2024/0215880 A1
(43) Pub. Date: Jul. 4, 2024

(54) SWITCH CHANNEL STAY-ON HEMOSHIELD CONNECTOR

(71) Applicant: CareFusion 303, Inc., San Diego, CA (US)

(72) Inventors: Mohammed Mehtab KHAN, Bengaluru (IN); Amarsinh Deeliprao JADHAV, Hyderabad (IN); Abin AUSTIN, Thrissur (IN)

(21) Appl. No.: 18/530,925

(22) Filed: Dec. 6, 2023

Related U.S. Application Data
(60) Provisional application No. 63/435,722, filed on Dec. 28, 2022.

(51) Int. Cl. A61B 5/15

(52) U.S. Cl. CPC A61B 5/150992; A61B 5/15003; A61B 5/150267; A61M 25/0097

(57) ABSTRACT

A fluid connector device for a blood collection system is disclosed. The including a fluid connector device may have passage for regulating movement of a fluid therethrough, including flow restriction devices having first and second passages that can regulate a fluid flow in a first direction through the device and can regulate a fluid flow in a second direction through the device and fluid flow restriction devices that can include a first connector having an internal surface defining an inner lumen, a second connector coupled to an end of the first connector, and a channel switching device contained within the lumen of the first and second connector.

### CROSS REFERENCE TO RELATED APPLICATION

This application claims the benefit of priority under 35 U.S.C. § 119 to U.S. Provisional Patent Application Ser. No. 63/435,722, entitled “SWITCH CHANNEL STAY-ON HEMOSHIELD CONNECTOR”, filed on Dec. 28, 2022, the entire contents of which is incorporated herein by reference.

### TECHNICAL FIELD

The present disclosure generally relates to blood draw and administration of parenteral fluids to a patient, and particularly to systems and methods to reduce hemolysis in PIVC blood draw.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used as an internal vacuum or a vacuum tube. The blood collection container may also be a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container.

The description provided in the background section should not be assumed to be prior art merely because it is mentioned in or associated with the background section. The background section may include information that describes one or more aspects of the subject technology.

### SUMMARY

The present disclosure provides devices and accessories for reducing hemolysis that can include features for restricting and regulating a fluid flow therethrough. In some instances, the present disclosure provides a multi-use connector with a switchable flow channel device (based on application) which is uniform and supports effective flushing of the connector.

In some instances, the present disclosure provides flow restriction devices that can regulate a fluid flow moving through the device in one or more direction, such as a first fluid flow moving in a direction away from a patient and a second fluid flow moving in a direction toward the patient.

The present disclosure also provides, in some embodiments, flow restriction devices that are configured to direct a fluid being withdrawn from a patient to move through a first passage, and to direct a fluid being infused toward the patient to move through any of a first and second passage.

The switchable flow channel device contains two paths or flow line, one is for infusion and the other for blood draw from a patient. This channel switch can be switched or rotated for a specific procedure to align with luers, for either the infusion or the blood draw procedure. Furthermore, the axis on which the channel switch device is rotated is asymmetrical, or off-center from the device. This asymmetrical rotational axis provides different advantages for each mode. The channel that is used for the blood draw mode is smaller in diameter to reduce hemolysis during blood draw, and the channel used for the infusion mode is larger in diameter to reduce instances of blockages during fluid infusion.

Embodiments of the present disclosure provide a fluid connector device, comprising a first connector, a second connector that combines with the first connector to form an internal chamber and a switchable flow channel device with a distal and a proximal end that is contained within the internal chamber. In some embodiments, the switchable flow channel device comprises two flow channels that are fluidly connected to the first connector and the second connector that allow for a fluid to flow in opposition directions through the switchable flow channel device. In some embodiments, the switchable flow channel device has two modes of use, an infusion mode and a blood draw mode. In some embodiments, the switchable flow channel device is manually rotated to switch between the infusion mode towards the blood draw mode. In some embodiments, the fluid flowing from the first connector to the second connector is an intravenous (IV) fluid. In some embodiments, the fluid flowing from the second connector to the first connector is blood. In some embodiments, the switchable flow channel device comprises a plurality of O-rings on the proximal and distal end of the switchable flow channel device.

In some embodiments, the fluid connector device 1 further comprises alignment posts extending from the first connector and the second connector that provide an axis that the switchable flow channel device can rotate around. In some embodiments, the alignment posts are not aligned in a center of the fluid connector device. In some embodiments, the axis on which switchable flow channel is rotated is asymmetrical, or off-center from the fluid connector device. In some embodiments, the flow channel that is used for the blood draw mode is smaller in diameter than the flow channel used in the infusion mode.

Embodiments of the present disclosure provide a blood collection system, comprising a blood collection device and a fluid connector device fluidly coupled to the blood collection device, the fluid connector device comprising a first connector, a second connector that combines with the first connector to form an internal chamber and a switchable flow channel with a distal and a proximal end that is in the internal chamber.

Embodiments of the present disclosure provide a method for using a fluid connector device comprising rotating a switchable flow channel device within the fluid connector device to switch between two modes of use, the fluid connector device comprising, a first connector and a second connector that combines with the first connector to form an internal chamber, wherein the switchable flow channel device comprises two flow channels that are fluidly connected to the first connector and the second connector that allow for a fluid to flow in opposition directions through the switchable flow channel device.

In some embodiments, the two modes of use are an infusion mode and a blood draw mode. In some embodiments, the fluid flowing from the first connector to the second connector is an intravenous (IV) fluid. In some embodiments, the fluid flowing from the second connector to the first connector is blood. In some embodiments, the switchable flow channel device comprises a plurality of O-rings on each of the proximal and distal ends of the switchable flow channel device. In some embodiments, the infusion mode is a default mode of the fluid connector device. In some embodiments, an axis on which the switchable flow channel device is rotated is spaced from the center of the fluid connector device. In some embodiments, the flow channel that is used for the blood draw mode is smaller in diameter than the flow channel used in the infusion mode. In some embodiments, when the o-rings are rotated into position and aligned in the fluid connector device, either in the infusion mode or the blood draw mode, the device will provide a tactile response to indicate to the user that the switchable flow channel device is ready for operation.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1A illustrates a vascular access device including a peripheral intravenous catheter (PIVC) assembly that includes a fluid connector device, in accordance with some embodiments of the present disclosure.

FIG. 1B illustrates a perspective exploded view of components of the fluid connector device of FIG. 1A, in accordance with some embodiments of the present disclosure.

FIG. 2A illustrates a perspective view of the fluid connector device of FIG. 1A in an infusion mode, in accordance with some embodiments of the present disclosure.

FIG. 2B illustrates a cross-sectional view of the fluid connector device of FIG. 2A, in accordance with some embodiments of the present disclosure.

FIG. 3A illustrates a perspective view of the fluid connector device of FIG. 2A rotated from the infusion mode towards a blood draw mode, in accordance with some embodiments of the present disclosure.

FIG. 3B illustrates a perspective view of the fluid connector device in the blood draw mode, in accordance with some embodiments of the present disclosure.

FIG. 3C illustrates a cross-sectional view of the fluid connector device of FIG. 3A, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing systems and methods to address hemolysis in PIVC blood draw with a hemolysis reduction accessory (also referred to herein as a fluid connector device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a fluid connector device) that regulates the overall flow rate of the entire fluid path as blood cells travel through. The fluid connector device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the fluid connector device and the blood collection device together. As such, this fluid connector device can be either for single blood draw or stay inline throughout indwell.

In accordance with various embodiments of the present disclosure, the fluid connector device may include a distal connector configured to couple to the catheter assembly. The distal connector may have a proximal end including a first connection portion, and a distal end including a second connection portion. The first connection portion may have an inner surface defining a lumen therethrough, and the second connection portion may have an inner surface defining a lumen therethrough. The distal connector may be in the form of a male luer connector or another suitable connector, which may be coupled with a female luer portion of the needleless connector which may be fluidly coupled to the catheter via the extension tubing.

In some embodiments, the fluid connector device may further include a proximal connector coupled to the proximal end of the distal connector. The proximal connector may be configured to couple to fluid collection device (e.g., a blood collection device). For example, the proximal connector may be integrated with the blood collection device or monolithically formed with the blood collection device as a single unit. As another example, the proximal connector may be in the form of a female luer connector or another suitable connector, which may be coupled with a male luer portion of the blood collection device. The proximal connector may have a proximal end, a distal end, and an internal surface defining a lumen extending therethrough for coupling to the male luer portion of the blood collection device.

In accordance with various embodiments of the present disclosure, the fluid connector device may further include an insert (channel switch) mounted in the lumen of the first connection portion and interposed between the proximal connector and the distal connector. As depicted, the insert may have an outer surface, a proximal end, and a distal end. The insert may further include a plurality of flow channels extending longitudinally from the proximal end to the distal end of the insert for selectively fluidly communicating the lumen of the first connection portion of the distal connector with the lumen of the proximal connector. In some embodiments, the plurality of channels may include a first or blood draw channel and a second or infusion channel. In some embodiments, the plurality of channels may include three or four channels for selective infusion or draw.

In some embodiments, the blood draw channel may be in the form of a micro-channel fluid pathway through which fluid (e.g., blood) flows from the distal connector to the proximal connector for collection in the fluid collection device. The blood draw channel in the form of a micro-channel along which fluid (e.g., blood) may flow from the distal connector into the proximal connector for collection into the fluid collection device, may have a diameter in the range of 0.015 inches to 0.15 inches, in some instances 0.02 inches to 0.1 inches, in other instances 0.04 inches to 0.08 inches, in other instances approximately 0.06 inches. Accordingly, during blood collection or withdrawal from the patient, the blood may flow into the blood collection device via the micro-channel defined in the blood flow channel having minimal diameter. The fluid connector device of the various embodiments described herein is advantageous over currently existing blood collection systems. For example, during blood draw with currently existing blood draw devices, blood cells may experience wall shear stress as they flow from the distal end to the proximal end of the blood collection systems. As previously described above, wall shear stress on blood cells is considered a major source of mechanical damage to blood cells causing hemolysis of the blood cells. The micro-channel defined in the blood draw channel having minimal diameter may facilitate increased flow resistance within the vascular access system to distribute the pressure differential and reduce shear stress experienced by the red blood cells of the blood. For example, the minimized diameter of the blood draw channel may provide increased resistance to flow of the blood and thereby decrease blood flow rate within the flow connector device. Since the decreased blood flow rate causes a reduction in shear stress experienced by the red blood cells of the blood, a risk of hemolysis during blood collection may advantageously be reduced.

Where the medical fluid is blood being withdrawn or collected from a patient, the medical fluid may be a blood sample, and the fluid collection device may be a blood collection device. In some embodiments, the blood collection device may be a luer lock access device (LLAD).

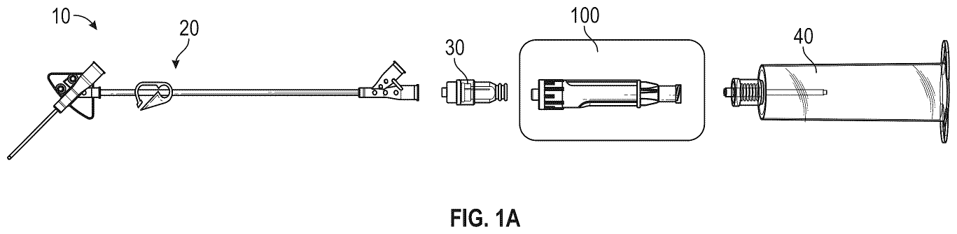

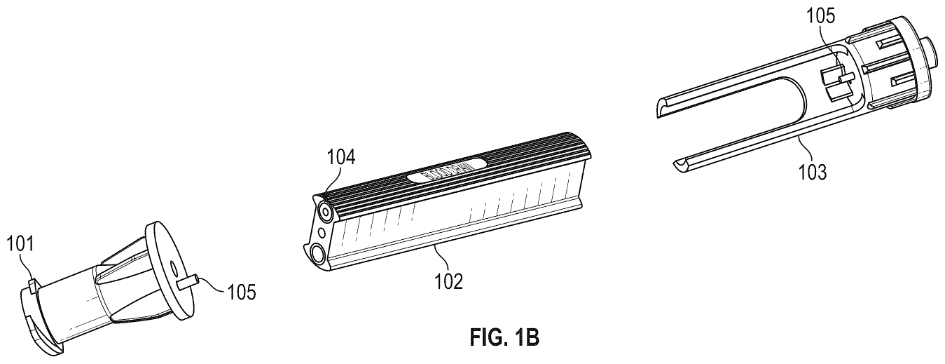

FIG. 1A illustrates a vascular access device including a peripheral intravenous catheter (PIVC) assembly that includes a fluid connector device, in accordance with some embodiments of the present disclosure. The fluid connector device may be configured to reduce a likelihood of hemolysis during blood collection using a vascular access device. In some embodiments, the vascular access device may include a catheter assembly (e.g., a PIVC). In some embodiments, the fluid connector device may include a distal end, which may include a body or distal connector configured to couple to the catheter assembly. The distal connector may include a male luer connector, or another suitable connector. In some embodiments, the catheter assembly may include a catheter hub, which may include a distal end, a proximal end, and a lumen extending through the distal end and the proximal end. The catheter assembly may further include a catheter, which may be secured within the catheter hub and may extend distally from the distal end of the catheter hub. In some embodiments, the catheter may be a peripheral intravenous catheter (PIVC).

FIG. 1A illustrates a collection system 10, in accordance with some aspects of the present disclosure. The collection system 10 is designed to collect fluid (e.g., blood) from a patient. Accordingly, the collection system 10 may be referred to as a blood collection system. The collection system 10 can take the form of a vascular access device with a PIVC assembly. As shown, the collection system 10 include a catheter assembly 20. In some embodiments, the catheter assembly 20 may include or correspond to any suitable catheter assembly. In some embodiments, the catheter assembly 20 may be integrated with and include an extension tube, which may extend from and be integrated with a side port of the catheter hub. A non-limiting example of an integrated catheter assembly is the BD NEXIVA™ Closed IV Catheter system, available from Becton Dickinson and Company. In some embodiments, a proximal end of the extension tube may be coupled to an adapter, such as, for example, a Y-adapter or single port luer adapter.

Also, the collection system 10 may include a connector 30 that connects to the catheter assembly 20. In order to collect fluid (e.g., blood) from a patient, the collection system 10 further includes a collector 40. When the collected fluid is blood from a patient, the collector 40 may be referred to as a blood collector.

Additionally, the collection system 10 includes a fluid connector device 100, in accordance with some aspects of the present disclosure. The fluid connector device 100 may be configured to reduce a likelihood of hemolysis during blood collection using the collection system 10. In some embodiments, the fluid connector device 100 may include a distal end, which may include a body or distal connector configured to couple to the catheter assembly 20 by way of the connector 30. The connector 30 may include a male luer 103 connector, or another suitable connector.

In some embodiments, the distal connector of fluid connector device 100 may be configured to couple to the Y-adapter of the catheter assembly 20 without the use of the connector 30. In some embodiments, the catheter assembly 20 may be non-integrated and may not include the extension tube. In these and other embodiments, the fluid connector device 100 may be configured to couple to the proximal end of the catheter hub or another suitable portion of the catheter assembly. In some embodiments, the catheter assembly 20 may be coupled to a removable extension tube. In some embodiments, the fluid connector device 100 may be coupled directly to the catheter adapter, eliminating the extension tube and providing a compact catheter system.

FIG. 1B illustrates an exploded view of the fluid connector device 100 shown in FIG. 1A, in accordance with some aspects of the present disclosure. The fluid connector device 100 may include a female luer 101 on the proximal end, a switchable flow channel (channel switch device) 102, and a male luer 103 on the distal end. The channel switch device may be altered based on the application of the fluid connector device and has a uniform channel, which supports effective flushing and reduces risk of hemolysis.

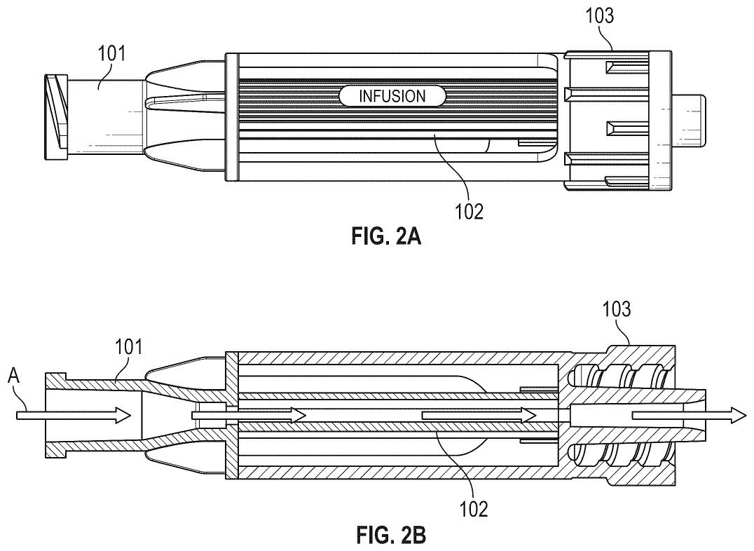

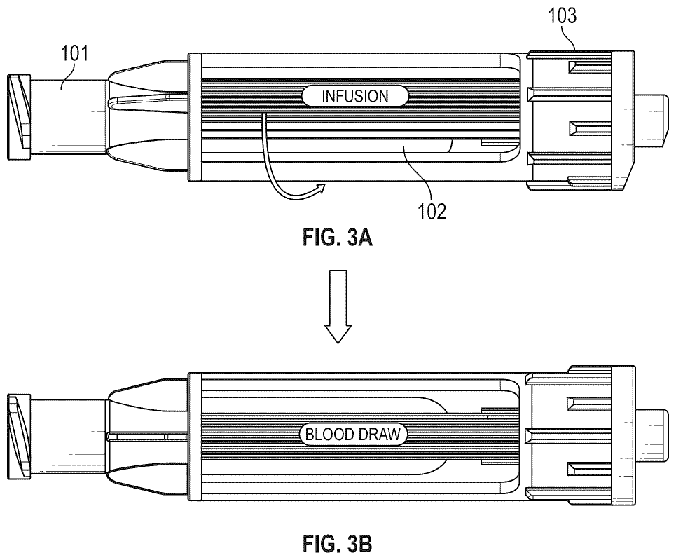

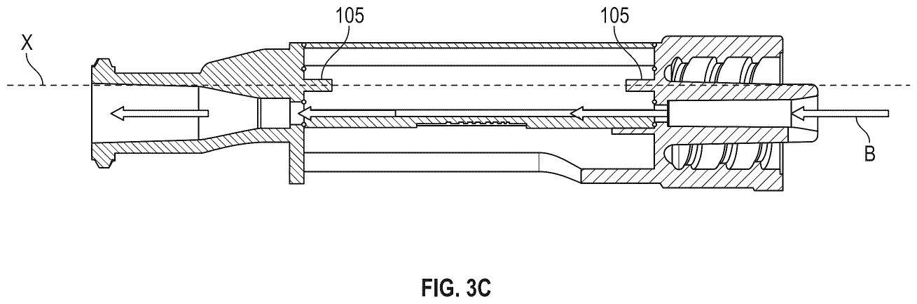

The channel switch device 102 may have a plurality of modes of operation. In accordance with FIGS. 2B and 3C, the channel switch may contain two paths or flow lines. In some embodiments, one flow line may be used for drug infusion (as shown by arrow A), and the other flow line is for blood draw (as shown by arrow B). This channel switch can be switched or rotated for a specific procedure to align with luers, be it infusion or blood draw procedure. As depicted in FIG. 3C, the axis X on which the channel switch device is rotated is asymmetrical, off-center, or spaced from the center of the device 102. In some embodiments, the rotational axis is centered on alignment posts 105 on both the female luer 101 and male luer 103. The alignment posts 105 provide an axis that the channel switch device can rotate around. As shown in FIG. 3C, alignment posts 105 are not in the center of the fluid connector device. In other embodiments, the alignment posts could be on the channel switch device. In some embodiments, the alignment post could extend through the fluid connector device. This asymmetrical rotational axis X provides different advantages for each mode. The channel that is used for the blood draw mode is smaller in diameter to reduce hemolysis during blood draw, and the channel used for the infusion mode is larger in diameter to prevent or limit the incidents of blockages during fluid infusion.

The default aligned channel is the infusion line. Whenever the blood draw procedure is required, the channel switch can be rotated and aligned with blood draw channel. In the drug infusion mode (arrow A), infusion flow-path is connected to male luer 103 and female luer 101. Diameter of this infusion flow-path is higher than the blood-draw flow path. In the blood draw mode (arrow B), blood-draw flow path is connected to male luer 103 and female luer 101. Diameter of this blood-draw flow path may be optimized for high quality blood draw.

In some embodiments, the channel switch device 102 may be labeled to assist the user in switching the channel for the proper procedure. As shown in FIGS. 3A-3B, the channel switch device 102 may be labeled with “INFUSION” to indicate that the drug infusion flow channel (arrow A) is open, or the channel switch device 102 may be labeled with “BLOOD DRAW” to indicate that the blood draw flow channel (arrow B) is open. In operation, the medical professional or other user may simply rotate the insert (switch channel) as illustrated in FIG. 3A to switch the fluid connector device 102 from the infusion mode illustrated in FIGS., 2A and 2B, to the blood draw mode illustrated in FIGS. 3B and 3C.

In order to prevent leakage in the flow channel, the channel switch device 102 may include a plurality of rings 104 mounted to the channel switch device on the proximal and distal ends of the channel switch device. In some embodiments, the rings 104 are O-rings.

The fluid connector device further comprises a tactile response configured to provide tactile feedback to a user of the fluid connector device when the o-rings on the channel switch device 102 are aligned in the fluid connector device 100. When the o-rings 104 are rotated into position, either in the infusion mode or the blood draw mode, the device will provide a tactile response to indicate to the user that the channel switch device is in place and ready for operation.

The fluid connector devices and associated blood collection systems of the various embodiments described herein additionally provide further advantages over currently existing blood collection systems. For example, add-on fluid connector devices described herein allow for hemolysis-reduction function to be integrated for PIVC blood draw. Further, the fluid connector devices described herein are compatible with PIVC placement and allow for seamless blood draw at insertion. Ion some embodiments, the fluid connector devices have the potential to stay inline throughout PIVC indwell for multiple blood draws. Additionally, since the fluid connector devices are an add-on which can be easily incorporated without any changes to existing PIVC, there is minimal impact to clinical setting and operations.

An optimized fluid pathway, also referred to herein as a first fluid pathway or flowpath or micro-channel, can be configured for providing a restricted flow rate for reducing hemolysis, and can have features including, but not limited to a tubular fluid pathway, a cannula, a lumen, a continuous non-linear channel, a groove, a fluid channel, and the like.

The fluid pathway can have a length that is selected based on one or more of the following: a gauge of a particular catheter, a particular catheter assembly configuration, or a clinical setup. In some embodiments, the optimized fluid pathway may include a length L from the female luer adapter 101 to the male luer adapter 103. In some embodiments, the optimized fluid pathway may include an inner diameter D.

Fluid flow in a tubular fluid pathway therethrough can be analyzed using Poiseuille's equation:

$$Q = \frac{π {D}^{4} Δ P}{128 μL} = \frac{Δ P}{{R}_{f}}$$

where ΔP is a change in pressure gradient across the length of the fluid pathway, D and L are the inner diameter and length, respectively, of the fluid pathway, μ is the viscosity of a fluid, and

$${R}_{f} = \frac{128 μL}{π {D}^{4}}$$

is the fluid resistance. Since μ is the viscosity of the fluid and not part of the extension tube geometry, a geometric factor G_{f} is defined such that R_{f} (the fluid resistance) is

$${R}_{f} = \frac{128 μ}{π} {G}_{f} , where {G}_{f} = \frac{L}{{D}^{4}} .$$

In some embodiments, the optimized fluid pathway may have multiple sections with lengths (L1, L2, L3) and inner diameters of (D1, D2, D3), the geometric factor is then:

$${G}_{f} = {\int }_{0}^{L} \frac{dl}{{D ( l )}^{4}}$$

In some embodiments, the optimized fluid pathway may have a cross section that is not circular or complicated inside diameter profile. The geometric factor can be determined by measuring the flow rate (Q) at given pressure (ΔP) with known viscosity (μ) fluid:

$${G}_{f} = \frac{πΔ P}{128 μ Q}$$

The G_{f} value of the optimized fluid pathway may be selected to reduce the max shear stress for each catheter gauge to be the same or less than the max shear stress of a BD 21G VACUTAINER®UltraTouch™ push button blood collection set, which was previously considered the gold standard for blood draws. In some embodiments, G_{f} value of the optimized fluid pathway may be selected to reduce the max shear stress for each catheter gauge to be the same or less than the max shear stress of a BD 25G VACUTAINER®UltraTouch™ push button blood collection set.

In some embodiments, and by way of non-limiting example, an optimized fluid pathway can have a diameter of approximately 0.014 inch. In another non-limiting example, a cross-sectional area of an optimized fluid pathway is approximately 0.000152 inch².

In some embodiments, the infusion channel may define a channel having a larger diameter than the blood draw channel through which fluid (e.g., infusion or intravenous (IV) fluid flows from the proximal connector to the distal connector for delivering the infusion fluid to the catheter via the extension tubing. In some embodiments, the second or infusion channel may have a diameter larger than the diameter of the micro-channel of the blood draw channel. For example, in some embodiments, the second or infusion channel may have a diameter approximately 4 or 5 times the size of the diameter of the micro-channel of the blood draw channel. The aforementioned configuration with the diameter of the infusion channel being larger than the diameter of the blood draw channel may be advantageous in further allowing an unrestricted and increased amount of the infusion fluid to flow to the patient in comparison to the smaller (minimized) diameter of the blood draw channel. Accordingly, the infusion fluid may flow in a second unrestricted (less flow resistance) direction (proximal to distal) opposite from the first direction (distal to proximal) in which the blood sample having blood cells flows.

The fluid connector devices and associated blood collection systems of the various embodiments described herein additionally provide further advantages over currently existing blood collection systems. For example, add-on fluid connector devices described herein allow for hemolysis-reduction function to be integrated for PIVC blood draw. Further, the fluid connector devices described herein are compatible with PIVC placement and allow for seamless blood draw at insertion. Furthermore, the fluid connector devices have the potential to stay inline throughout PIVC indwell for multiple blood draws. Additionally, since the fluid connector devices are an add-on which can be easily incorporated without any changes to existing PIVC, there is minimal impact to clinical setting and operations.

In some embodiments, the catheter assembly may include or correspond to any suitable catheter assembly. In some embodiments, the catheter assembly may be integrated and include an extension tube, which may extend from and be integrated with a side port 59 of the catheter hub. A non-limiting example of an integrated catheter assembly is the BD NEXIVA™ Closed IV Catheter system, available from Becton Dickinson and Company. In some embodiments, a proximal end of the extension tube may be coupled to an adapter, such as, for example, a Y-adapter or single port luer adapter. In some embodiments, the distal connector of fluid connector device may be configured to couple to the Y-adapter.

In some embodiments, the catheter assembly may be non-integrated and may not include the extension tube. In these and other embodiments, the fluid connector device may be configured to couple to the proximal end of the catheter hub or another suitable portion of the catheter assembly. In some embodiments, the catheter assembly may be coupled to a removable extension tube. In some embodiments, the fluid connector device may be coupled directly to the catheter adapter, eliminating the extension tube and providing a compact catheter system.

In some embodiments, the male luer 103 and female luer 101 may be joined together by ultrasonic welding. In the fully assembled flow connector device, the channel switch device is contained within the male and female luers.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

### Illustration of Subject Technology as Clauses

Clause 1: A fluid connector device, comprising: a first connector; a second connector that combines with the first connector to form an internal chamber; and a switchable flow channel device with a distal and a proximal end that is contained within the internal chamber.

Clause 2: The fluid connector device of clause 1, wherein the switchable flow channel device comprises two flow channels that are fluidly connected to the first connector and the second connector that allow for a fluid to flow in opposition directions through the switchable flow channel device.

Clause 3: The fluid connector device of clause 1, wherein the switchable flow channel device has two modes of use, an infusion mode and a blood draw mode.

Clause 4: The fluid connector device of clause 3, wherein the switchable flow channel device is manually rotated to switch between the infusion mode towards the blood draw mode.

Clause 5: The fluid connector device of clause 1, wherein the fluid flowing from the first connector to the second connector is an intravenous (IV) fluid.

Clause 6: The fluid connector device of clause 1, wherein the fluid flowing from the second connector to the first connector is blood.

Clause 7: The fluid connector device of clause 1, wherein the switchable flow channel device comprises a plurality of O-rings on the proximal and distal end of the switchable flow channel device.

Clause 8: The fluid connector device of clause 1 further comprising alignment posts extending from the first connector and the second connector that provide an axis that the switchable flow channel device can rotate around.

Clause 9: The fluid connector device of clause 8, wherein the alignment posts are not aligned in a center of the fluid connector device.

Clause 10: The fluid connector device of clause 8, wherein the axis on which switchable flow channel is rotated is asymmetrical, or off-center from the fluid connector device.

Clause 11: The fluid connector device of claim 3, wherein the flow channel that is used for the blood draw mode is smaller in diameter than the flow channel used in the infusion mode.

Clause 12: A blood collection system, comprising: a blood collection device; and a fluid connector device fluidly coupled to the blood collection device, the fluid connector device comprising: a first connector; a second connector that combines with the first connector to form an internal chamber; and a switchable flow channel with a distal and a proximal end that is in the internal chamber.

Clause 13: A method for using a fluid connector device comprising: rotating a switchable flow channel device within the fluid connector device to switch between two modes of use, the fluid connector device comprising: a first connector; and a second connector that combines with the first connector to form an internal chamber, wherein the switchable flow channel device comprises two flow channels that are fluidly connected to the first connector and the second connector that allow for a fluid to flow in opposition directions through the switchable flow channel device.

Clause 14: The method of clause 13, wherein the two modes of use are an infusion mode and a blood draw mode.

Clause 15: The method of clause 13, wherein the fluid flowing from the first connector to the second connector is an intravenous (IV) fluid.

Clause 16: The method of clause 13, wherein the fluid flowing from the second connector to the first connector is blood.

Clause 17: The method of clause 13, wherein the switchable flow channel device comprises a plurality of O-rings on each of the proximal and distal ends of the switchable flow channel device.

Clause 18: The method of clause 14, wherein the infusion mode is a default mode of the fluid connector device.

Clause 19: The method of clause 13, wherein an axis on which the switchable flow channel device is rotated is spaced from the center of the fluid connector device.

Clause 20: The method of clause 13, wherein the flow channel that is used for the blood draw mode is smaller in diameter than the flow channel used in the infusion mode

Clause 21: The method of clause 17, wherein when the o-rings are rotated into position and aligned in the fluid connector device, either in the infusion mode or the blood draw mode, the device will provide a tactile response to indicate to the user that the switchable flow channel device is ready for operation.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector device, comprising:
a first connector;
a second connector that combines with the first connector to form an internal chamber; and
a switchable flow channel device with a distal and a proximal end that is contained within the internal chamber.

2. The fluid connector device of claim 1, wherein the switchable flow channel device comprises two flow channels that are fluidly connected to the first connector and the second connector that allow for a fluid to flow in opposition directions through the switchable flow channel device.

3. The fluid connector device of claim 1, wherein the switchable flow channel device has two modes of use, an infusion mode and a blood draw mode.

4. The fluid connector device of claim 3, wherein the switchable flow channel device is manually rotated to switch between the infusion mode towards the blood draw mode.

5. The fluid connector device of claim 1, wherein the fluid flowing from the first connector to the second connector is an intravenous (IV) fluid.

6. The fluid connector device of claim 1, wherein the fluid flowing from the second connector to the first connector is blood.

7. The fluid connector device of claim 1, wherein the switchable flow channel device comprises a plurality of O-rings on the proximal and distal end of the switchable flow channel device.

8. The fluid connector device of claim 1 further comprising alignment posts extending from the first connector and the second connector that provide an axis that the switchable flow channel device can rotate around.

9. The fluid connector device of claim 8, wherein the alignment posts are not aligned in a center of the fluid connector device.

10. The fluid connector device of claim 8, wherein the axis on which switchable flow channel is rotated is asymmetrical, or off-center from the fluid connector device.

11. The fluid connector device of claim 3, wherein the flow channel that is used for the blood draw mode is smaller in diameter than the flow channel used in the infusion mode.

12. A blood collection system, comprising:
a blood collection device; and
a fluid connector device fluidly coupled to the blood collection device, the fluid connector device comprising:
a first connector;
a second connector that combines with the first connector to form an internal chamber; and
a switchable flow channel with a distal and a proximal end that is in the internal chamber.

13. A method for using a fluid connector device comprising:
rotating a switchable flow channel device within the fluid connector device to switch between two modes of use, the fluid connector device comprising:
a first connector; and
a second connector that combines with the first connector to form an internal chamber,
wherein the switchable flow channel device comprises two flow channels that are fluidly connected to the first connector and the second connector that allow for a fluid to flow in opposition directions through the switchable flow channel device.

14. The method of claim 13, wherein the two modes of use are an infusion mode and a blood draw mode.

15. The method of claim 13, wherein the fluid flowing from the first connector to the second connector is an intravenous (IV) fluid.

16. The method of claim 13, wherein the fluid flowing from the second connector to the first connector is blood.

17. The method of claim 13, wherein the switchable flow channel device comprises a plurality of O-rings on each of the proximal and distal ends of the switchable flow channel device.

18. The method of claim 13, wherein an axis on which the switchable flow channel device is rotated is spaced from the center of the fluid connector device.

19. The method of claim 13, wherein the flow channel that is used for the blood draw mode is smaller in diameter than the flow channel used in the infusion mode.

20. The method of claim 17, wherein when the o-rings are rotated into position and aligned in the fluid connector device, either in the infusion mode or the blood draw mode, the device will provide a tactile response to indicate to the user that the switchable flow channel device is ready for operation.
